(19) United States
(12) Patent Application Publication
(10) Pub. No.: US 2023/0035485 A1
(43) Pub. Date: Feb. 2, 2023

(54) IRRIGATION BASIN

(71) Applicant: Raymond K. WURAPA, Blacklick, OH (US)

(72) Inventor: Raymond K. WURAPA, Blacklick, OH (US)

(21) Appl. No.: 17/444,067

(22) Filed: Jul. 30, 2021

(51) Int. Cl. A61G 13/10

(52) U.S. Cl. CPC A61G 13/102; A61B 2217/007

(57) ABSTRACT

An irrigation basin includes a base, a wall extending upwardly from the base and having opposed aligned first wall portions defining outwardly extending generally horizontal contoured recesses adapted to receive corresponding portions of an extremity, opposed aligned second wall portions separating the recesses, each second wall portion extending to a contoured top surface, the recesses further extending downward forming opposed buttresses each terminating in a plane coplanar with the planar termination of the foot. At least one opening is formed in at least one upper surface adapted to receive a surgical device, and at least one groove is formed in at least one second portion extending from the top surface to the upper surface of the base. A post extends from the upper surface of the base, the groove adapted to receive a suction device, the post and corresponding portions of the groove adapted to capture the suction device.

### FIELD OF THE INVENTION

The present invention is directed to an irrigation basin for treating injured extremities.

### BACKGROUND OF THE INVENTION

Medical treatment of injured extremities requires providing a stable support for the extremity, while simultaneously providing access to the extremity by medical professionals, as well as catching spillage of bodily fluids resulting from the injury, and permitting irrigation of the extremity during treatment. Furthermore, there are challenges associated with surgical devices (e.g., misplacement, impediment), experienced during their intermittent utilization during treatment.

There is a need in the art for an irrigation basin that helps address such challenges to medical treatment.

### SUMMARY OF THE INVENTION

In one embodiment, an irrigation basin includes a base defining a closed geometry and having a peripheral vertical foot extending from a lower surface of the base and terminating in a plane, the upper surface of the base being nonplanar. The irrigation basin further includes a wall extending upwardly from the base opposite the foot, opposed aligned first wall portions defining outwardly extending generally horizontal contoured recesses adapted to receive corresponding portions of an extremity, opposed aligned second wall portions separating the recesses, each second wall portion extending to a contoured top surface, the recesses further extending downward forming opposed buttresses each terminating in a plane coplanar with the planar termination of the foot. The irrigation basin further includes at least one opening formed in at least one upper surface adapted to receive a surgical device. The irrigation basin further includes at least one groove formed in at least one second portion extending from the top surface to the upper surface of the base. The irrigation basin further includes a post extending from the upper surface of the base, the groove adapted to receive a suction device, and the post and corresponding portions of the groove adapted to capture an end of the suction device.

Other features and advantages of the present invention will be apparent from the following more detailed description, taken in conjunction with the accompanying drawings which illustrate, by way of example, the principles of the invention.

### BRIEF DESCRIPTION OF THE DRAWINGS

FIG. 1 is an upper perspective view of an exemplary irrigation basin.

FIG. 2 is a lower perspective view of the irrigation basin of FIG. 1 rotated clockwise about a vertical axis.

FIG. 3 is a front elevation view of the irrigation basin of FIG. 1.

FIG. 4 is a side elevation view of the irrigation basin of FIG. 1.

FIG. 5 is a top view of the irrigation basin of FIG. 1.

FIG. 6 is a bottom view of the irrigation basin of FIG. 1.

FIG. 7 is a cross sectional view taken along line 7-7 of FIG. 1.

FIG. 8 is a cross sectional view taken along line 8-8 of FIG. 1.

FIG. 9 is a cross sectional view taken along line 9-9 of FIG. 1.

FIG. 10 is a cross sectional view taken along line 10-10 of FIG. 1.

FIG. 11 is a cross sectional view taken along line 11-11 of FIG. 1.

Wherever possible, the same reference numbers will be used throughout the drawings to represent the same parts.

### DETAILED DESCRIPTION OF THE INVENTION

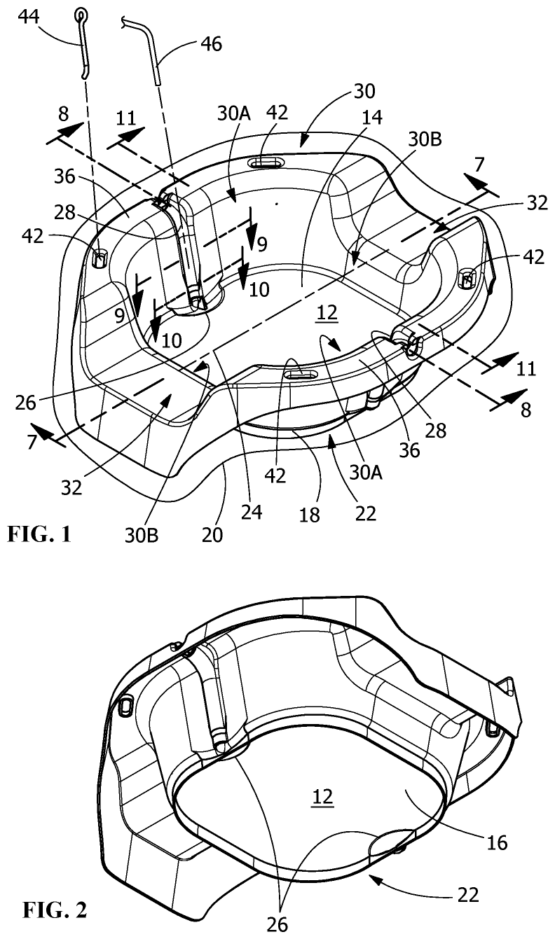

FIGS. 1-10 collectively show different views of an exemplary irrigation basin 10 of the present invention. More specifically, as shown in FIG. 1. irrigation basin 10 includes a base 12 defining a closed geometry, such as generally defining a rectangle such as a square, such as resembling a square with rounded corners. The generally square shape provides a stable geometry to help prevent lateral tipping of the irrigation basin during use. In other embodiments, base 12 may define other closed geometries, such as, but not limited to closed regular shapes (i.e., symmetrical shapes such as symmetrical about an axis 24) although suitable closed irregular shapes (i.e., asymmetrical shapes) may be utilized. Base 12 includes an upper surface 12 and a lower surface 16 (FIG. 2) from which a peripheral vertical foot 18 terminating in a plane 22 that is supported by a support surface 20 such as a table top. Upper surface 14 of base 12 is preferably nonplanar such as defining a convex surface to help channel fluids for collection. In one embodiment, upper surface 14 may be planar, but is non-horizontal, so as to permit fluids draining onto the upper surface to be collected from one or more positions such as along the periphery of the base. More specifically, one or more regions of upper surface 14 of base 12 surrounding a groove 28 formed in a wall 30 extending upwardly from base 12 defines a well 26 (only one well 26 shown in FIG. 1) that is positioned vertically below a remaining portion of the upper surface of base 12.

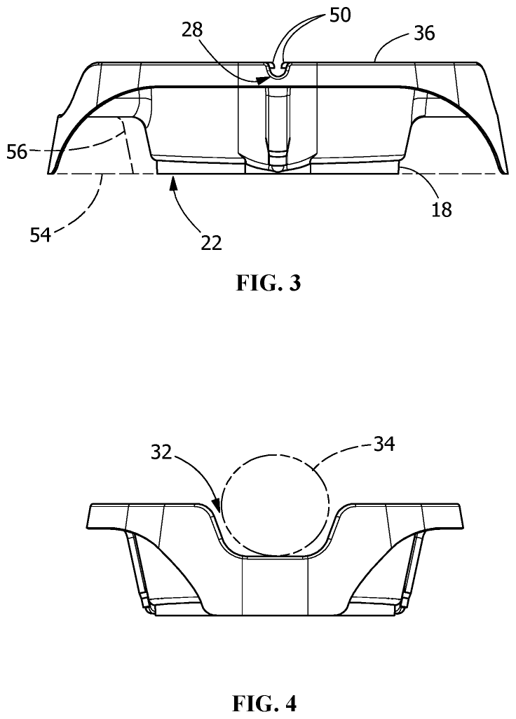

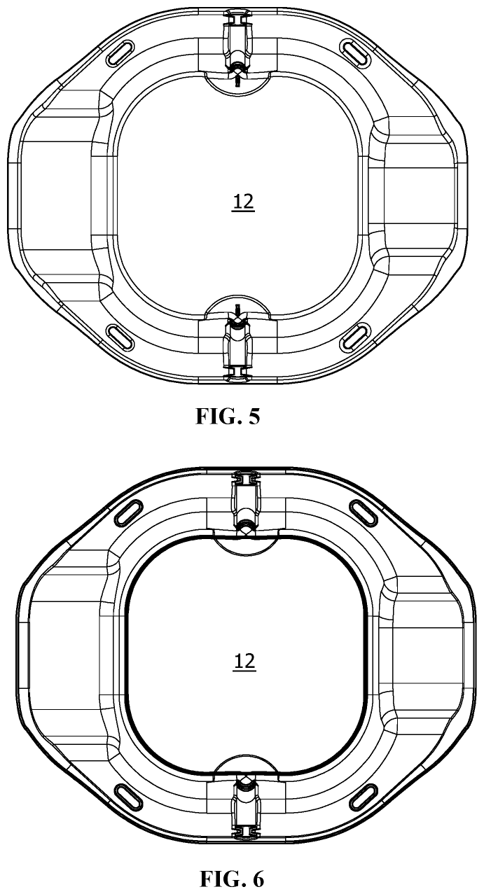

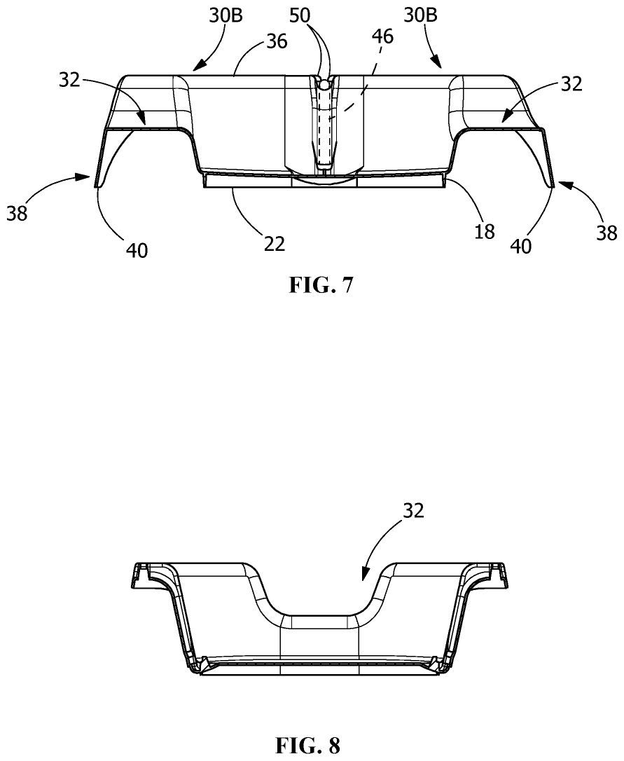

As further shown in FIG. 1, wall 30 extends upwardly from base 12 opposite foot 18, with opposed aligned wall portions 30B defining outwardly extending generally horizontal contoured recesses 32 (FIG. 4) adapted to receive corresponding portions of an extremity 34 (FIG. 4). As further shown in FIG. 1, opposed aligned wall portions 30A separate recesses 32, each wall portion 30A extends to a contoured top surface 36. At least one opening 42 is formed in at least one top surface 36 that is adapted to receive a surgical device 44 such as a hemostat, which not only acts to help prevent misplacement of the surgical device, but opening 42 may be adapted such as with a slot and/or at least partial recessing relative to the surrounding top surface 36 in order to reduce the opportunity of the surgical device “snagging” or otherwise interfering with administration of medical treatment. Opening(s) 42 may be all the same size or sized differently. As shown in FIG. 7, recesses 32 further extend downward forming opposed buttresses 38, each buttress 38 terminating in a plane 40 that is coplanar with the planar termination of foot 18 to provide enhanced stability of the irrigation basin.

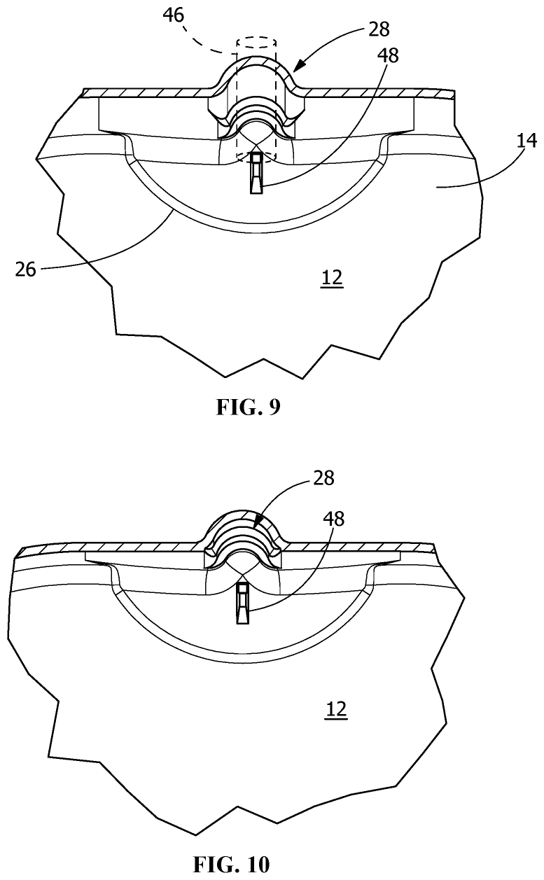

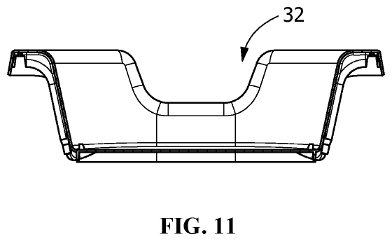

Returning to FIG. 1, at least one groove 28 is formed in at least one wall portion 30A extending from the top surface 36 to well 26 formed in upper surface 14 of base 12 such as for receiving a suction device 46 or instrument. Groove 28 is preferably sized along its length to receive suction device 46 so that suction device 46 is at nearly flush or even recessed relative to the surrounding surface of wall portion 30A. To help secure suction device 46 in its most effective installed position with the tip of the suction device 46 positioned in close proximity to upper surface 14 of base 12, and more preferably the tip of the suction device 46 protruding into well 26, as shown in FIG. 9, a post 48 extends from upper surface 14 of base 12, the groove adapted to receive a suction device 46, and post 48 and corresponding portions of groove 28 adapted to capture an end or tip of suction device 46. To further help secure suction device 46, a securement device 50 such as a pair of tabs or protrusions 52 extending toward one another is formed in top surface 36 in close proximity of groove 28 (FIG. 3 shows securement device 50 prior to installation of suction device 46; FIG. 7 shows securement device 50 with suction device 46 installed). Stated another way, groove 28 comprises a securement device 50 formed in top surface 26.

In one embodiment, as shown in FIG. 3, each top surface 36 further extends downwardly forming a skirt having continuous peripheral buttress 54 (only one portion of the continuous buttress 54 shown) with the opposed buttresses 38 (FIG. 7) extending from the recesses, the continuous buttress 54 (FIG. 3) terminating in a plane coplanar with the termination plane 22 of foot 18.

In one embodiment, as shown in FIG. 3, each top surface 36 further extends downwardly forming a discontinuous peripheral buttress 56 (only one portion of the discontinuous buttress 56 shown) with the opposed buttresses 38 (FIG. 7) extending from the recesses, the discontinuous buttress 56 (FIG. 3) terminating in a plane coplanar with the termination plane 22 of foot 18. In one embodiment, the discontinuous buttress 56 may define a scalloped shape.

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. An irrigation basin comprising:
a base defining a closed geometry and having a peripheral vertical foot extending from a lower surface of the base and terminating in a plane, the upper surface of the base being nonplanar;
a wall extending upwardly from the base opposite the foot, opposed aligned first wall portions defining outwardly extending generally horizontal contoured recesses adapted to receive corresponding portions of an extremity, opposed aligned second wall portions separating the recesses, each second wall portion extending to a contoured top surface, the recesses further extending downward forming opposed buttresses each terminating in a plane coplanar with the planar termination of the foot;
at least one opening formed in at least one upper surface adapted to receive a surgical device;
at least one groove formed in at least one second portion extending from the top surface to the upper surface of the base;
a post extending from the upper surface of the base, the groove adapted to receive a suction device, and the post and corresponding portions of the groove adapted to capture an end of the suction device.

2. The irrigation basin of claim 1, wherein a region of the upper surface of the base surrounding the groove defines a well that is vertically below a remainder of the upper surface.

3. The irrigation basin of claim 1, wherein the closed geometry of the base generally defines a square.

4. The irrigation basin of claim 1, wherein the groove comprises a securement device formed in the top surface.

5. The irrigation basin of claim 4, wherein the securement device comprises a pair of protrusions extending toward one another.

6. The irrigation basin of claim 1, wherein each top surface further extends downwardly forming a continuous peripheral buttress with the opposed buttresses extending from the recesses, the continuous buttress terminating in a plane coplanar with the planar termination of the foot.

7. The irrigation basin of claim 1, wherein each top surface further extends downwardly forming a pair of opposed second buttress adjacent the opposed buttresses extending from the recesses, the opposed buttress pair and the opposed second buttresses pair each terminating in a plane coplanar with the planar termination of the foot.

8. The irrigation basin of claim 1, wherein each top surface further extends at least partially downwardly forming a discontinuous skirt between each corresponding buttress.

9. The irrigation basin of claim 1, wherein the upper surface of the base defines a convex surface.
